(19) United States
(12) Patent Application Publication
(10) Pub. No.: US 2021/0364347 A1
(43) Pub. Date: Nov. 25, 2021

(54) VIBRONIC MULTISENSOR

(71) Applicant: Endress+Hauser SE+Co. KG, Maulburg (DE)

(72) Inventors: Sergey Lopatin, Lörrach (DE); Jan Schleiferböck, Rümmingen (DE); Tobias Brengartner, Emmendingen (DE)

(21) Appl. No.: 17/291,177

(22) PCT Filed: Jun. 5, 2019
(86) PCT No.: PCT/EP2019/064724

§ 371 Date: May 4, 2021

(30) Foreign Application Priority Data
Nov. 5, 2018 (DE) 10 2018 127 526.9

(51) Int. Cl. G01H 11/08; H04B 11/00; C12M 1/34

(52) U.S. Cl. CPC G01H 11/08; C12M 41/00; H04B 11/00

(57) ABSTRACT

Disclosed is a method for determining and/or monitoring at least two different process variables of a medium, wherein a sensor unit is excited to vibrate mechanically by means of an excitation signal, the mechanical vibrations are received from the sensor unit and are converted into a first reception signal, the sensor unit emits a transmission signal and receives a second reception signal, and a first process variable is determined on the basis of the first reception signal and a second process variable is determined on the basis of the second reception signal. Disclosed also is an apparatus configured to carry out a method according to the invention.

The invention relates to a method for determining and/or monitoring at least two different process variables of a medium by means of an apparatus comprising a sensor unit having at least one mechanical-vibration-capable unit and a first and a second piezoelectric element. The invention further relates to an apparatus configured to carry out a method according to the invention. The medium is located in a container, e.g., in a reservoir or in a pipeline.

Vibronic sensors are often used in process and/or automation technology. In the case of fill-level measuring devices, they have at least one mechanical-vibration-capable unit, such as a tuning fork, a single rod, or a membrane. During operation, the mechanical-vibration-capable unit is excited to vibrate mechanically by means of a driving/receiving unit, often in the form of an electromechanical transducer unit that can, in turn, be a piezoelectric drive or an electromagnetic drive, for example. A wide variety of corresponding field devices are produced by the applicant and are distributed under the name LIQUIPHANT or SOLIPHANT, for example. The underlying measurement principles are known in principle from numerous publications. The driving/receiving unit excites the mechanical-vibration-capable unit to vibrate mechanically by means of an electrical excitation signal. Conversely, the driving/receiving unit can receive the mechanical vibrations of the mechanical-vibration-capable unit and convert them into an electrical reception signal. The driving/receiving unit is accordingly either a separate driving unit and a separate receiving unit, or a combination driving/receiving unit.

The driving/receiving unit is usually part of an electrical feedback resonant circuit, by means of which the mechanical-vibration-capable unit is excited to vibrate mechanically. For example, the resonant circuit condition according to which the amplification factor is ≥1 and all phases occurring in the resonant circuit result in a multiple of 360° must be fulfilled for a resonant vibration. For excitation and fulfillment of the resonant circuit condition, a specific phase shift between the excitation signal and the reception signal must be ensured. A specifiable value for the phase shift, i.e., a target value for the phase shift between the excitation signal and the reception signal, is therefore frequently set. For this purpose, various solutions, both analog and digital methods, have become known from the prior art, as described, for example, in documents DE102006034105A1, DE102007013557A1, DE102005015547A1, DE102009026685A1, DE102009028022A1, DE102010030982A1, or DE00102010030982A1.

Both the excitation signal and the reception signal are characterized by their frequency ω, amplitude A and/or phase Φ. Accordingly, changes in these variables are typically used to determine the respective process variables. The process variable may, for example, be a fill level, a specified fill level, or the density or the viscosity of the medium, and also the flow rate. In the case of a vibronic level switch for liquids, for example, a distinction is made between whether the vibration-capable unit is covered by the liquid or vibrates freely. The two states, the free state and the covered state, are, for example, distinguished on the basis of different resonant frequencies, i.e., on the basis of a frequency shift.

The density and/or viscosity, in turn, can only be determined with such a measuring device if the vibration-capable unit is covered by the medium. In connection with the determination of the density and/or viscosity, different possibilities have likewise become known from the prior art, such as those disclosed in documents DE10050299A1, DE102007043811A1, DE10057974A1, DE102006033819A1, DE102015102834A1, or DE102016112743A1.

With a vibronic sensor, a plurality of process variables can be determined accordingly and used for characterizing the respective process. In many cases, however, further information about the process, especially, knowledge of further physical and/or chemical process variables and/or process parameters, is required for comprehensive process monitoring and/or control. This can be achieved, for example, by integrating further field devices into the respective process. The measured values provided by the various measuring devices can then be further processed in a suitable manner in a unit superordinate to the devices.

However, it is now the case that the different measuring devices have different measurement accuracies on the one hand. In addition, drift and/or aging effects can respectively be very different. However, such effects can make the respective measurement or process monitoring and/or control considerably more difficult or imprecise. In addition, it may be difficult to respectively determine the respective state of the individual field devices during continuous operation.

The object of the present invention is thus to expand the functionality of a vibronic sensor. This object is achieved by the method according to claim 1 and by the apparatus according to claim 13.

With regard to the method, the object is achieved by a method for determining and/or monitoring at least two different process variables of a medium, wherein
a sensor unit is excited to vibrate mechanically by means of an excitation signal,
the mechanical vibrations are received by the sensor unit and converted into a first reception signal,
the sensor unit emits a transmission signal and receives a second reception signal, and
a first process variable is determined on the basis of the first reception signal and a second process variable is determined on the basis of the second reception signal.

The sensor unit is part of an apparatus for determining and/or monitoring at least two different process variables of a medium and comprises a mechanical-vibration-capable unit along with at least a first and a second piezoelectric element. The mechanical-vibration-capable unit is, for example, a membrane, a single rod, an arrangement of at least two vibrating elements, or a tuning fork. In addition, the two piezoelectric elements can serve at least partially as a driving/receiving unit for generating the mechanical vibrations of the mechanical-vibration-capable unit.

The transmission signal can furthermore be emitted by one of the two piezoelectric elements and received by the respective other piezoelectric element in the form of the second reception signal. The transmission signal passes through the medium at least temporarily and in sections and is influenced by the physical and/or chemical properties of the medium and can accordingly be used for determining a second process variable of the medium.

Within the scope of the present invention, it is advantageously possible to implement at least two measurement principles in a single apparatus. The sensor unit carries out mechanical vibrations on the one hand; in addition, a transmission signal is emitted. In response to the mechanical vibrations and to the transmission signal, two reception signals are received and evaluated with regard to at least two different process variables. The two reception signals can advantageously be evaluated independently of one another. In this way, according to the invention, the number of determinable process variables can be significantly increased, which results in a higher functionality of the respective sensor or in an extended field of application.

In one embodiment of the invention, the excitation signal and the transmission signal are simultaneously supplied to the sensor unit, wherein the excitation signal and the transmission signal are superimposed on one another. Alternatively, however, the excitation signal and the transmission signal can also be alternately supplied to the sensor unit.

In a preferred embodiment, the excitation signal is an electrical signal having at least one specifiable frequency, especially, a sinusoidal or a rectangular signal. The mechanical-vibration-capable unit is preferably excited at least temporarily to vibrate resonantly. The mechanical vibrations are influenced by the medium surrounding the vibration-capable unit, so that conclusions regarding different properties of the medium are possible on the basis of a reception signal representing the vibrations.

In another particularly preferred embodiment, the transmission signal is an ultrasonic signal, especially, a pulsed ultrasonic signal, especially, at least one ultrasonic pulse. An ultrasound-based measurement is accordingly carried out within the scope of the present invention as the second measurement method used. The transmission signal emitted in each case at least partially passes through the medium and is influenced by the latter in its properties. Accordingly, conclusions on different media can likewise be drawn on the basis of the respectively received second reception signal.

With the two methods used, it is advantageously possible to determine at least partially different process variables and/or process parameters independently of one another with one apparatus, so that a comprehensive analysis of the respective process is made possible by means of a single measuring device. In addition, by using the same sensor unit for both measurement methods, the accuracy of the measurements can be significantly increased. In addition, the two process variables can be used to monitor the state of the apparatus. Numerous embodiments are possible in this regard, some preferred variants of which are given below.

In a particularly preferred embodiment, the first process variable is the density of the medium and the second process variable is the sound velocity within the medium or a variable derived therefrom. The density of the medium is thus determined on the basis of the first reception signal and the sound velocity within the medium is thus determined on the basis of the second reception signal.

In this case, it is advantageous if a reference value for the density is determined on the basis of the sound velocity, and wherein the reference value is compared by means of a value for the density determined from the first reception signal. A concentration of a reference substance dissolved in a reference medium in a specifiable reservoir is preferably determined on the basis of the sound velocity determined from the second reception signal. The reference value for the density of the reference medium can subsequently be determined from the concentration. Moreover, a measured value for the density can be determined from the first reception signal. The two values for the density can then be compared with one another. The value for the density determined from the first reception signal can especially be adjusted on the basis of the reference value for the density determined from the second reception signal. In this way, an adverse influence on the geometry of the respectively used container on the vibronic determination of the density can be compensated for.

In addition, it is advantageous if at least a third process variable, especially, the viscosity, of the medium is determined. However, it goes without saying that, in addition to the process variables explicitly mentioned here, further process variables and/or process parameters that are accessible by means of the two measurements carried out can also be determined and used for characterizing the respective process.

In one embodiment of the method, it is determined on the basis of the first and second reception signals and/or on the basis of the first and second process variables whether a deposit has formed on the sensor unit. The two reception signals usually each behave differently depending on a deposit in the region of the sensor unit. The presence of a deposit can accordingly be ascertained, for example, on the basis of a temporal consideration of the two reception signals and/or process variables.

In another embodiment of the method, a drift and/or aging of the sensor unit is determined on the basis of the first and second reception signals and/or on the basis of the first and second process variables. In this connection as well, a temporal consideration of the first and second reception signals and/or of the first and second process variables can be carried out, for example.

A particularly preferred embodiment provides for the first and second reception signals, the first and second process variables and/or a time profile of the first and second reception signals and/or of the first and second process variables to be compared with one another. The presence of a deposit, a drift or aging of the sensor unit can then be inferred from the comparison. Since at least two reception signals or process variables are accessible, a high degree of accuracy with regard to the statements made in each case about a deposit, a drift or aging can be achieved.

By implementing according to the invention two different measurements with a single sensor unit, the presence of a deposit, or a drift or aging of the sensor unit can accordingly be reliably detected.

In another particularly preferred embodiment, an influence of a deposit, a drift and/or aging of the sensor unit on the first and/or the second reception signal is reduced or compensated in the determination and/or monitoring of at least one process variable or in the determination of a variable derived from at least one process variable and/or from at least one reception signal. Accordingly, the influence of a deposit, a drift and/or aging of the sensor unit can be taken into account in the determination and/or monitoring of the respective process variable, so that the respective process variable can be determined without on the presence of a deposit, a drift and/or aging. In order to reduce or compensate for the influence, a suitable algorithm can, for example, be stored, with the aid of which a value that is not falsified by the influence of the deposit, the drift and/or aging of the sensor unit can be determined for the respective process variable. Improved measurement accuracy can thus be achieved.

In yet another particularly preferred embodiment, a first concentration of a first substance contained in the medium and a second concentration of a second substance contained in the medium are determined on the basis of the first and second reception signals and/or on the basis of the first and second process variables. According to the prior art, for such an analysis of the medium with respect to two different substances, two separate measuring devices that provide different measurands are usually required. According to the invention, by contrast, a statement about two different components in a medium can be reliably made by means of a single apparatus.

A preferred use of the method relates to the monitoring of a fermentation process. In fermentation, sugar is converted to ethanol. In order to be able to ensure qualitative monitoring, it is therefore necessary to determine the concentration of both sugar and ethanol. This is possible within the framework of the present invention.

The object underlying the invention is furthermore achieved by an apparatus for determining and/or monitoring a first and a second process variable of a medium, which apparatus is configured to carry out a method according to at least one of the described embodiments.

It is advantageous if the sensor unit comprises a mechanical-vibration-capable unit and at least a first piezoelectric element, especially, at least a first and a second piezoelectric element. However, more than two piezoelectric elements that may be arranged at different positions relative to the vibration-capable unit may also be present.

Thus, in a preferred embodiment, the mechanical-vibration-capable unit is a tuning fork with a first and a second vibrating element, wherein the first piezoelectric element is at least partially arranged in one of the two vibrating elements, wherein, especially, the first piezoelectric element is at least partially arranged in the first vibrating element and the second piezoelectric element is at least partially arranged in the second vibrating element. Corresponding embodiments of a sensor unit have been described, for example, in the documents DE102012100728A1 and in the previously unpublished German patent application with reference number DE102017130527A1. Both applications are referred to in their entirety within the framework of the present invention. It should be pointed out that the present invention is, however, not limited to one of the possible embodiments of the sensor unit described in the two documents. These are only exemplary possible structural embodiments of the sensor unit that are suitable for carrying out the method according to the invention. For example, the use of a single piezoelectric element, which can be arranged, for example, in one of the two vibrating elements, is also sufficient. It is also not absolutely necessary to arrange the piezoelectric elements exclusively in the region of the vibrating elements. Rather, individual piezoelectric elements used may also be arranged in the region of the membrane or in further vibrating elements, which are not used for the vibronic excitation and which are likewise applied to the membrane.

It should also be pointed out that the embodiments described in connection with the method according to the invention can also be applied mutatis mutandis to the apparatus according to the invention and vice versa.

The invention is explained in greater detail with reference to the following figures. The following is shown:

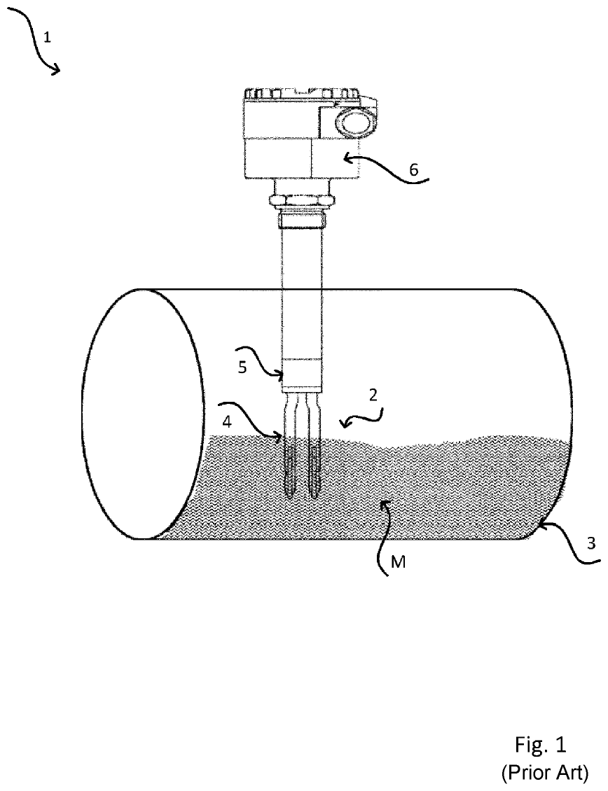

FIG. 1: a schematic drawing of a vibronic sensor according to the prior art,

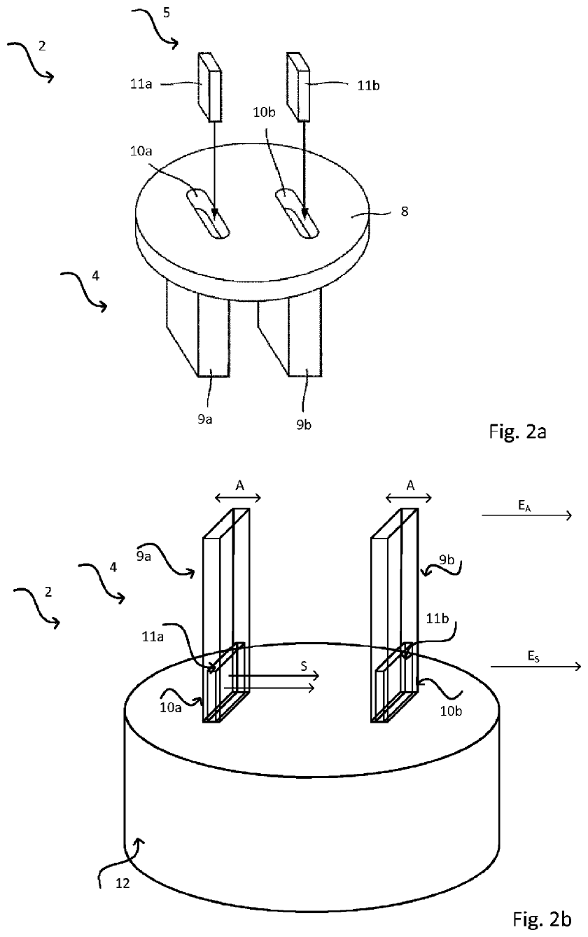

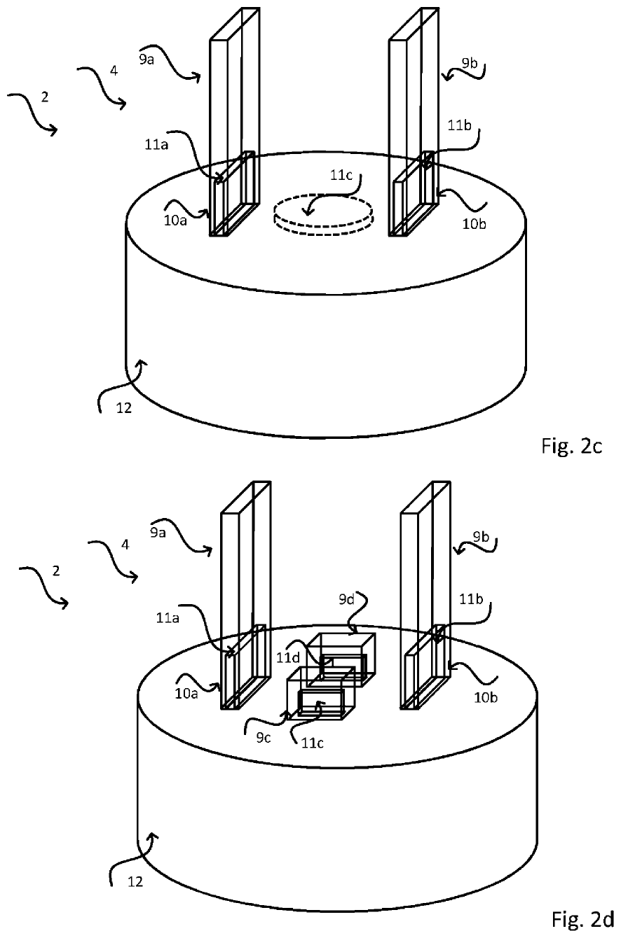

FIG. 2 a plurality of possible embodiments of a sensor unit that are known per se from the prior art and are suitable for carrying out the method according to the invention, and

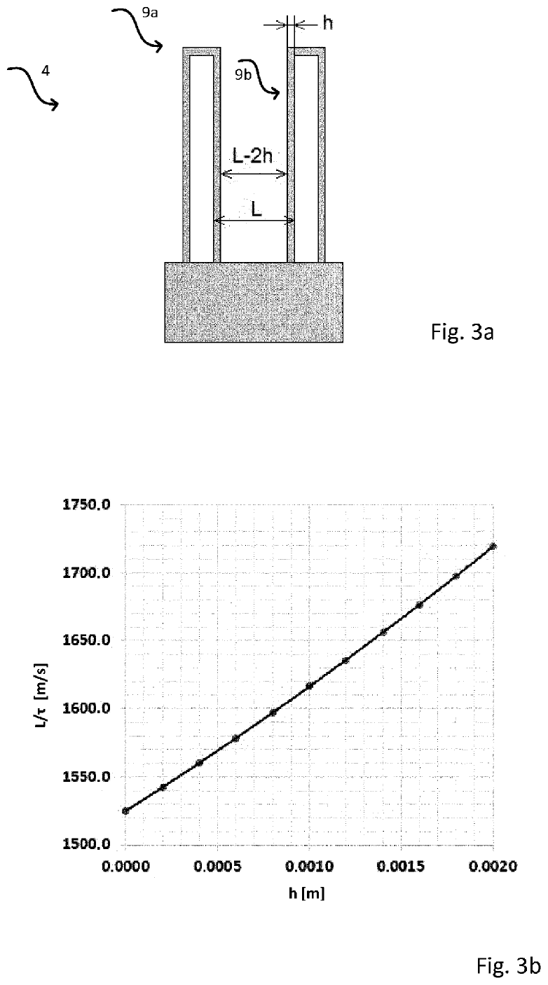

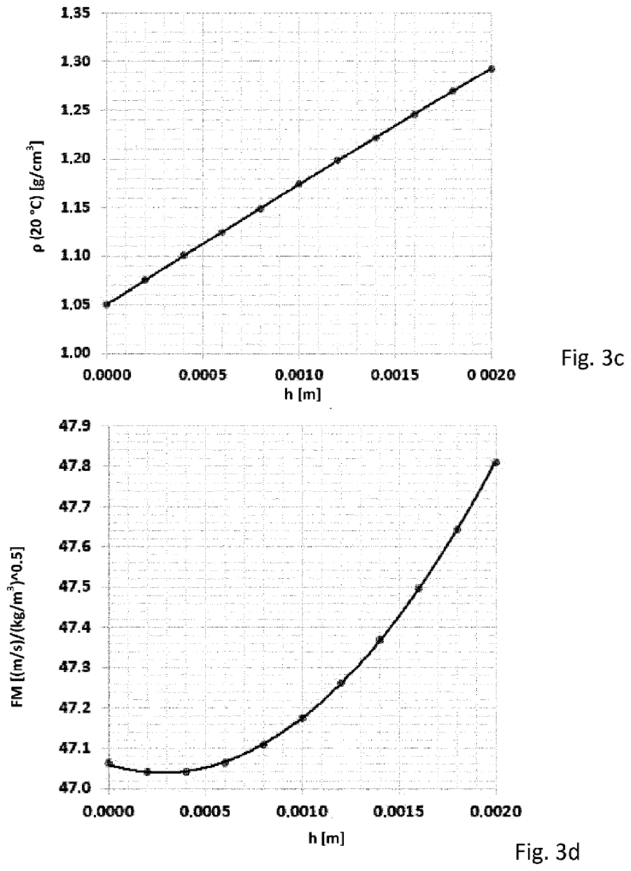

FIG. 3 an illustration of an embodiment of the method according to the invention for detecting deposits in the region of the sensor unit.

In the figures, identical elements are respectively provided with the same reference signs.

FIG. 1 shows a vibronic sensor 1 having a sensor unit 2. The sensor has a mechanical-vibration-capable unit 4 in the form of a tuning fork, which is partially dipped into a medium M, which is located in a reservoir 3. The vibration-capable unit 4 is excited by the excitation/receiving unit 5 to vibrate mechanically and can, for example, be by means of a piezoelectric stack drive or bimorphic drive. Other vibronic sensors have, for example, electromagnetic driving/receiving units 5. It is possible to use a single driving/receiving unit 5, which serves to excite the mechanical vibrations and to detect them. However, it is also conceivable to implement one each, a driving unit and a receiving unit. FIG. 1 furthermore shows an electronic unit 6, by means of which the signal acquisition, evaluation and/or feeding takes place.

FIG. 2 shows, by way of example, various sensor units 2, which are suitable for carrying out a method according to the invention. The mechanical-vibration-capable unit 4 shown in FIG. 2a comprises two vibrating elements 9a, 9b, which are mounted on a base 8 and which are therefore also referred to as fork teeth. Optionally, a paddle may respectively also be formed on the end sides of the two vibrating elements 9a, 9b [not shown here]. In each of the two vibrating elements 9a, 9b, a cavity 10a, 10b, especially, a pocket-like cavity, is respectively introduced, in which at least one piezoelectric element 11a, 11b of the driving/receiving unit 5 is respectively arranged. Preferably, the piezoelectric elements 11a and 11b are embedded in the cavities 10a and 10b. The cavities 10a, 10b can be such that the two piezoelectric elements 11a, 11b are located completely or partially in the region of the two vibrating elements 9a, 9b. Such an arrangement along with similar arrangements are extensively described in DE102012100728A1.

Another possible exemplary embodiment of a sensor unit 2 is depicted in FIG. 2b. The mechanical-vibration-capable unit 4 has two vibrating elements 9a, 9b, which are aligned in parallel to one another and are configured here in a rod-shaped manner. They are mounted on a disk-shaped element 12 and can be excited separately from one another to vibrate mechanically. Their vibrations can likewise be received and evaluated separately from one another. The two vibrating elements 9a and 9b respectively have a cavity 10a and 10b, in which at least one piezoelectric element 11a and 11b is respectively arranged in the region facing the disk-shaped element 12. With respect to the embodiment according to FIG. 2b, reference is again furthermore made to the previously unpublished German patent application with reference number DE102017130527A1.

As shown schematically in FIG. 2b, according to the invention, the sensor unit 2 is supplied on the one hand with an excitation signal A in such a way that the vibration-capable unit 4 is excited to vibrate mechanically. The vibrations are generated by means of the two piezoelectric elements 11a and 11b. It is conceivable both for both piezoelectric elements to be supplied with the same excitation signal A and for the first vibrating element 11a to be supplied with a first excitation signal A₁ and the second vibrating element 11b to be supplied with a second excitation signal A₂. It is also conceivable for a first reception signal E_{A} to be received on the basis of the mechanical vibrations, or for each vibrating element 9a, 9b to receive a separate reception signal E_{A1} or E_{A2}.

In addition, a transmission signal S is emitted from the first piezoelectric element 11a and is received in the form of a second reception signal E_{S} by the second piezoelectric element 11b. Since the two piezoelectric elements 11a and 11b are arranged at least in the region of the vibrating elements 9a and 9b, the transmission signal S passes through the medium M, provided that the sensor unit 2 is in contact with the medium M and is influenced accordingly by the properties of the medium M. The transmission signal S is preferably an ultrasonic signal, especially, a pulsed ultrasonic signal, especially, at least one ultrasonic pulse. However, it is also conceivable for the transmission signal S to be emitted by the first piezoelectric element 11a in the region of the first vibrating element 9a and to be reflected at the second vibrating element 9b. In this case, the second reception signal E_{S} is received by the first piezoelectric element 11a. In this case, the transmission signal S passes through the medium M twice, which leads to a doubling of a transit time T of the transmission signal S.

In addition to these two embodiments shown of an apparatus 1 according to the invention, numerous other variants are also conceivable, which likewise fall within the present invention. For example, for the embodiments according to figures FIG. 2a and FIG. 2b, it is possible to use only one piezoelectric element 11a, 11b and to arrange it at least in one of the two vibrating elements 9a, 9b. In this case, the piezoelectric element 9a serves to generate the excitation signal and the transmission signal S, and to receive the first E₁ and the second reception signal E₂. In this case, the transmission signal is reflected at the second vibrating element 9b without piezoelectric element 11b.

Another exemplary possibility is depicted in FIG. 2c. Here, a third piezoelectric element 11c is provided in the region of the membrane 12. The third piezoelectric element 11c serves to generate the excitation signal A and to receive the first reception signal E₁; the first 11a and the second piezoelectric element 11b serve to generate the transmission signal S or to receive the second reception signal E₂. Alternatively, it is possible, for example, to generate the excitation signal A and the transmission signal S and receive the second reception signal E₂ with the first 11a and/or the second piezoelectric element 11b, wherein the third piezoelectric element 11c serves to receive the first reception signal E₁. It is also possible to generate the transmission signal S with the first 11a and/or the second piezoelectric element 11b and the excitation signal A with the third piezoelectric element 11c and to receive the first E₁ and/or the second reception signal E₂ with the first 11a and/or the second piezoelectric element 11b. In the case of FIG. 2c, it is also possible for other embodiments to dispense with the first 11a or the second piezoelectric element 11b.

Yet another possible embodiment of the apparatus 1 is the subject matter of FIG. 2d. Starting from the embodiment of FIG. 2b, the apparatus comprises a third 9c and a fourth vibrating element 9d. However, the latter do not serve to generate vibrations. Rather, a third 11c and a fourth piezoelectric element 11d are respectively arranged in the additional elements 9c, 9d. In this case, the vibronic measurement is carried out by means of the first two piezoelectric elements 11a, 11b and the ultrasonic measurement by means of the other two piezoelectric elements 11c, 11d. Here as well, a piezoelectric element, e.g., 11b and 11d, can be dispensed with depending on the measurement principle. For reasons of symmetry, however, it is advantageous to always use two additional vibrating elements 9c, 9d.

In principle, the first E_{A} and the second reception signal E_{S} result according to the invention from different measurement methods and can be evaluated independently of one another with respect to different process variables P₁ and P₂. This results in a higher degree of accuracy with regard to the determination of the various available process variables and in a greater number of determinable variables. A comprehensive and precise characterization of the respective process is accordingly possible.

An advantageous embodiment of the method according to the invention includes the determination of the concentration of two different substances contained in the medium. In order to be able to determine a first concentration of a first substance and a second concentration of a second substance, which are both contained in the same medium, two different process variables or process parameters must be determined independently of one another. According to the invention, the two necessary process variables or process parameters can be determined by means of two independent measurement methods, but by means of the same sensor unit. This leads to increased accuracy with regard to the determination of the two concentrations and.

A preferred application in this context consists in monitoring a fermentation process. In this case, sugar is converted to ethanol. An exemplary possibility for determining the two concentrations and of sugar and ethanol consists in determining the density p of the medium M on the basis of the first reception signal and the sound velocity of the medium on the basis of the second reception signal.

Another preferred application consists in monitoring an inversion of sugar or an invert sugar. In this case, the proportion to which a sugar mixture, generally household sugar, has converted to glucose or fructose is monitored. In this case, the two concentrations of glucose and fructose can also be determined on the basis of the first and second reception signals.

The density p can be determined, for example, on the basis of the following equation:

$$ρ = \frac{1}{S} \cdot [ {( \frac{{F}_{0}}{{F}_{M e d}} )}^{2} - 1 ]$$

Here, F_{Med} is the vibration frequency of the vibration-capable unit 4 in the medium M, F₀ is the reference frequency of the vibration-capable unit 4 in vacuum or in air, and S describes the sensitivity of the sensor unit 2. The vibration frequency of the vibration-capable unit 4 in the medium M, F_{Med}, can be determined directly on the basis of the first reception signal E_{A}.

The sound velocity v_{M} of the medium M can in turn be determined from the distance L between the first 11a and the second piezoelectric element 11b, which serve as transmitting unit and receiving unit, along with the transit time T of the transmission signal S from the first 11a to the second piezoelectric element 11b according to the following equation:

$${v}_{M} = \frac{L}{τ}$$

The dependence of the density p and the sound velocity v_{M} is illustrated in FIG. 3a. FIG. 3a shows for this purpose a schematic drawing of a vibration-capable unit 4 in the form of a tuning fork with two vibrating elements 9a and 9b arranged at a distance L from one another. For the following consideration, it is furthermore assumed that a deposit of thickness h has formed in the region of the vibrating elements 9a and 9b.

FIG. 3b shows the sound velocity v_{M}, which was calculated on the basis of the measured transit time T and on the basis of the distance L between the two vibrating elements 9a and 9b, for a medium M with a density p of 2.0 g/cm³ at a temperature of 20° C. As the deposit increases, or as the thickness h of the deposit increases, the measured sound velocity v_{M} increases.

FIG. 3c again shows the density p, calculated on the basis of the measured vibration frequency f of the vibration-capable unit 4, at a temperature of 20° C. as a function of the deposit thickness h. The density p also increases with increasing thickness h of the deposit, but the slopes of the density p and of the sound velocity v_{M} are respectively different depending on the thickness h of the deposit.

Hereinafter, a preferred exemplary embodiment for compensating or reducing the influence of a deposit on the determination of a process variable P₁-P₃ is illustrated. The following considerations apply analogously to the case in which a drift and/or aging of the sensor unit 2 occurs. It should furthermore be pointed out that the compensation of the influence of the deposit described here is merely one of many ways of compensating for the influence of a deposit. Accordingly, the present invention is by no means limited to the exemplary embodiment indicated below.

In order to compensate for the influence of a deposit, a variable FM derived from at least one process variable can be determined. In the present case, the variable FM is determined on the basis of the sound velocity v_{M} and the density p according to

$$F M = \frac{{v}_{m}}{{ρ}^{n}}$$

This variable is shown in FIG. 3d for the case of n=0.5. As can be seen from the graph, the influence of a deposit of thickness h on the variable FM is negligible. If a process variable P₁-P₃ is calculated on the basis of the variable FM, the calculation takes place essentially without the influence of a deposit.

### LIST OF REFERENCE SIGNS

1 Vibronic sensor
2 Sensor unit
3 Reservoir
4 Vibration-capable unit
5 Driving/receiving unit
6 Electronic unit
8 Base
9a, 9b Vibrating elements
10a, 10b Cavities
11a 11b Piezoelectric elements
12 Disk-shaped element
M Medium
P₁-P₃ Process variables
A Excitation signal
S Transmission signal
E_{A} First reception signal
E_{S} Second reception signal
ΔΦ Specifiable phase shift
p Density of the medium
v Viscosity of the medium
v_{M} Sound velocity of the medium
T Transit time
a First substance
b Second substance
Cₐ Concentration of the first substance
C_{b} Concentration of the second substance
L Distance between the two fork teeth
H Thickness of the deposit on the sensor unit

1-16. (canceled)

17. A method for determining and/or monitoring at least two different process variables of a medium, comprising:
exciting a sensor unit to vibrate mechanically by means of an excitation signal;
receiving mechanical vibrations by the sensor unit and converting the mechanical vibrations into a first reception signal;
emitting from the sensor unit a transmission signal and receiving a second reception signal; and
determining a first process variable on the basis of the first reception signal and determining a second process variable on the basis of the second reception signal.

18. The method according to claim 17,
wherein the sensor unit is simultaneously supplied with the excitation signal and with the transmission signal and the excitation signal and the transmission signal are superimposed on one another, or
wherein the sensor unit is alternately supplied with the excitation signal and with the transmission signal.

19. The method according to claim 17,
wherein the excitation signal is an electrical signal having at least one specifiable frequency, including a sinusoidal or a rectangular signal.

20. The method according to claim 17,
wherein the transmission signal is an ultrasonic signal, including a pulsed ultrasonic signal having at least one ultrasonic pulse.

21. The method according to claim 17,
wherein the first process variable is a density of the medium and the second process variable is a sound velocity within the medium or a variable derived from the sound velocity within the medium.

22. The method according to claim 21, further comprising:
determining a reference value for the density on the basis of the sound velocity; and
comparing the reference value with the density determined from the first reception signal.

23. The method according to claim 17, further comprising:
determining at least a third process variable of the medium.

24. The method according to claim 17, further comprising;
determining on the basis of the first reception signal and the second reception signal and/or on the basis of the first process variable and the second process variable whether a deposit has formed on the sensor unit.

25. The method according to claim 17, further comprising:
determining a drift and/or an aging of the sensor unit on the basis of the first and the second reception signal and/or on the basis of the first and the second process variable.

26. The method according to claim 17, further comprising:
comparing with one another the first and the second reception signal, the first and the second process variable and/or a time profile of the first and the second reception signal and/or of the first and the second process variable.

27. The method according to claim 17,
wherein in the determination and/or monitoring of at least one process variable or in the determination of a variable derived from at least one process variable and/or from at least one reception signal, an influence of a deposit, a drift and/or aging of the sensor unit on the first and/or the second reception signal is reduced or compensated for.

28. The method according to claim 17, further comprising:
determining a first concentration of a first substance contained in the medium and a second concentration of a second substance contained in the medium on the basis of the first and the second reception signal and/or on the basis of the first and the second process variable.

29. A use of the method according to at least one of the preceding claims for monitoring a fermentation process or for monitoring an inversion of sugar.

30. An apparatus for determining and/or monitoring a first and a second process variable of a medium, wherein the apparatus is configured to:
excite a sensor unit to vibrate mechanically by means of an excitation signal;
receive mechanical vibrations by the sensor unit and convert the mechanical vibrations into a first reception signal;
emit from the sensor unit a transmission signal and receive a second reception signal; and
determine a first process variable on the basis of the first reception signal and determine a second process variable on the basis of the second reception signal.

31. The apparatus according to claim 30,
wherein the sensor unit includes a mechanical-vibration-capable unit and at least a first piezoelectric element and a second piezoelectric element.

32. The apparatus according to claim 31,
wherein the mechanical-vibration-capable unit is a tuning fork having a first and a second vibrating element,
wherein the first piezoelectric element is at least partially arranged in one of the two vibrating elements, and
wherein the first piezoelectric element is at least partially arranged in the first vibrating element and the second piezoelectric element is at least partially arranged in the second vibrating element.
